(19) United States
(12) Patent Application Publication
(10) Pub. No.: US 2003/0044795 A1
(43) Pub. Date: Mar. 6, 2003

(54) Biochemical markers of the human endometrium

(76) Inventors: Inger Byrajalsen, Hoersholm (DK); Peter M. Larsen, Odense (DK); Stephen J. Fey, Aarhus (DK)

Correspondence Address: PENNIE & EDMONDS LLP, 1667 K STREET NW, SUITE 1000, WASHINGTON, DC, 20006

(21) Appl. No.: 09/935,642

(22) Filed: Aug. 24, 2001

Related U.S. Application Data
(63) Continuation-in-part of application No. 09/254,472, filed on Oct. 27, 1999, now abandoned.
Application No. 09/254,472 is a 371 of international application No. PCT/GB97/02394, filed on Sep. 5, 1997.

(30) Foreign Application Priority Data
Sep. 6, 1996 (GB) 9618600.2
Apr. 8, 1997 (GB) 9707132.8

(51) Int. Cl. C12Q 1/68; G01N 33/53; G01N 33/557

(52) U.S. Cl. 435/6; 435/7.1; 436/517

(57) ABSTRACT

Assay methods are provided for detection or quantitation of any of several proteins which are specifically produced in the endometrium in association with hyperplasia, adenocarcinoma or the proliferative phase of the endometrium. The relevant proteins have been identified by 2D gel electrophoresis with subsequent sequence identification by mass spectroscopic finger printing of tryptic digests.

The endometrium is the mucous lining of the uterine cavity. During the menstrual cycle, the endometrium is the organ in the body that shows the greatest changes under the influence of the sex hormones, oestradiol and progesterone. In the oestrogen dominated phase the endometrium proliferates until progesterone from the corpus luteum transforms the oestrogen-primed proliferative endometrium to a secretory phase endometrium. In due course this is followed by shedding of the fully transformed endometrium during the menstruation, and a new cycle will begin.

Persistent unbalanced oestrogen stimulation either due to increased endogenous production of oestrogens, or replacement therapy in which oestrogens are given alone, is associated with increased risk of developing endometrial hyperplasia and subsequently endometrial adenocarcinoma. Histologically, these pathological conditions are characterised by increased thickness of the endometrium and irregular pattern of the endometrial glandular cells.

Endometrial adenocarcinoma is a life threatening condition.

At present the endometrial status is assessed by histological and biochemical analysis of endometrial biopsies. This is time-consuming, expensive and causes discomfort for the woman. It would be highly desirable to identify biochemical markers which could be measured in body fluids reflecting the endometrial status, obviating the need for endometrial biopsies. The detection of such markers in histological samples would also however be advantageous as an additional method of recognising the histological status of such samples.

We have now discovered that certain proteins are produced in the endometrium in increased amounts associated with hyperplasia and that certain proteins are produced in increased amounts associated with adenocarcinoma. These two groups of proteins overlap somewhat. The present invention relates in a first aspect to such proteins and to their diagnostic uses.

Unless otherwise indicated, references to the proteins herein include references to modified forms of the proteins and derivatives of the proteins, including but not restricted to glycosylated, phosphorylated, acetylated, methylated or lipidated forms thereof.

Thus the invention provides a method of characterising a biological sample comprising detecting or quantitating therein one or more proteins produced by the endometrium in increased amounts in hyperplasia or in adenocarcinoma as shown by 2D gel electrophoresis comparison of cell lysates of endometrial biopsies from normal endometrium and endometrium showing hyperplasia or adenocarcinoma, excluding variations due to the menstrual cycle, or detecting or quantitating a fragment or breakdown product thereof, or a nucleic acid coding therefor, or an antibody thereto.

The invention includes a method of characterising a biological sample comprising detecting or quantitating therein one or more proteins produced by the endometrium in increased amounts in hyperplasia or in adenocarcinoma and characterised by one of the following combinations of molecular weight and pI values:

hyperplasia

| pI | MW kDa |
|---|---|
| 6.7 | 91 |
| 6.6 | 90 |
| 6.9 | 64 |
| 6.6 | 67 |
| 6.3 | 66 |
| 6.8 | 46 |
| 5.7 | 41 |
| 5.5 | 35 |
| 5.3 | 13 |
| 6.6 | 101 |
| 5.8 | 14 |
| 7.4 | 51 |
| 8.2 | 44 |
| 9.5 | 48 |

adernocarcinoma

| pI | MW (kDa) |
|---|---|
| 6.3 | 32 |
| 6.0 | 109 |
| 6.7 | 91 |
| 6.6 | 90 |
| 6.9 | 64 |
| 6.6 | 67 |
| 6.3 | 66 |
| 6.2 | 62 |
| 6.2 | 45 |
| 5.7 | 45 |
| 5.4 | 33 |
| 6.3 | 27 |
| 6.5 | 103 |
| 6.8 | 90 |
| 6.9 | 78 |
| 5.3 | 13 |
| 6.2 | 130 |
| 6.3 | 66 |
| 6.3 | 73 |
| 8.3 | 32 |
| 8.1 | 55 |
| 8.2 | 44 |
| 6.6 | 111 |
| 7.7 | 43 |
| 9.5 | 48 |
| 8.3 | 32 |
| 7.7 | 39 |

or a fragment or breakdown product thereof, or a nucleic acid coding therefor, or an antibody thereto.

Said protein, fragment, breakdown product, antibody or nucleic acid may preferably be detected in a body fluid sample but may also be detailed in other forms of sample such as histological samples or cytological samples.

The invention includes an immunological binding partner specifically reactive with a protein as defined above with a fragment or breakdown product thereof or with a nucelic acid coding therefor.

It also includes a cell line producing a monoclonal antibody being such an immunological binding partner.

The invention includes also an assay kit for use in such an analysis method comprising an immunological binding partner as described.

This aspect of the invention has resulted from studies aiming to detect endometrial proteins with increased synthesis in endometrial adenocarcinoma as compared to the synthesis during the normal menstrual cycle; to detect endometrial proteins with increased synthesis in endometrial hyperplasia as compared to the synthesis during the normal menstrual cycle; and to detect proteins showing a cycle-related expression during the normal menstrual cycle.

In a second aspect the invention relates to the discovery of markers of the “proliferative” phase of the human endometrium. A protein marker for the “secretory” phase of the endometrium has been previously described, see U.S. Pat. No. 4,489,166. No similar marker has been described for the proliferative phase although certain candidate proteins were described in Ref. 1.

Under influence of the sex hormones, oestradiol and progesterone, the human endometrium undergoes cyclical variation with an oestrogen-dominated phase, i.e. the proliferative phase, an ovulation phase, i.e. the interval phase, a progesterone-dominated phase, i.e. the secretory phase, and finally the endometrium is shed, i.e. the menstrual phase. The same cyclical variation of the endometrium is seen in postmenopausal women receiving sequentially combined hormone replacement therapy. The demand for endometrial status assessment has highly increased in the latest decade, not only on account of the extensive research into fertility, but also in order to estimate endometrial response to the large number of combined oestrogens/progestogen preparations used in hormone replacement therapy. It would be highly desirable to identify biochemical markers which could be measured in body fluids reflecting the endometrial status, obviating the need for endometrial biopsies. Studies have suggested that serum placental protein 14 (PP14), which is produced in the glandular cells of the secretory phase endometrium (Ref. 3), is a reliable marker of the secretory phase endometrium. It has been shown that serum PP14 strongly correlates with the secretory activity of the endometrium in postmenopausal women receiving hormone replacement therapy (Ref. 4,5). No similar marker exists for the proliferative phase endometrium.

We have now discovered that certain proteins are produced in the endometrium in increased amounts in proliferative phase endometrium as compared to secretory phase endometrium.

According to this aspect of the invention there is now provided a method of characterising a biological sample comprising detecting or quantitating therein one or more proteins produced by the endometrium in increased amounts during the proliferative phase of the endometrium as shown in 2D gel elctrophoresis comparison of cell lysates of endometrial biopsies from normal endometrium in its proliferative and secretory phases and characterised by one of the following combinations of molecular weight and pI values:

| pI | MW (kDa) |
|---|---|
| 6.9 | 86 |
| 5.4 | 34 |
| 5.6 | 67 |
| 5.3 | 23 |
| 6.8 | 52 |
| 8.7 | 47 |
| 8.2 | 138 |
| 6.5 | 124 |
| 7.7 | 119 |
| 7.8 | 119 |
| 8.1 | 66 |
| 7.1 | 59 |
| 6.8 | 66 |
| 7.9 | 48 |
| 7.7 | 31 |
| 6.8 | 29 |
| 7.2 | 70 |
| 8.0 | 119 |
| 6.7 | 62 |

or a fragment or breakdown product thereof, or a nucleic acid coding therefor or an antibody thereto.

Such a method may preferably be for detecting the phase of the endometrium.

The preferred features of the first aspect of the invention apply also to this second aspect.

This aspect of the invention includes a method of determining the proliferative/secretory phase status of the endometrium comprising the quantitative or qualitative measurement in a sample of any one or more of the proteins defined above or a breakdown product or fragment thereof. It also includes an immunological binding partner for any of the said proteins, breakdown products or fragments or a cell line producing such a binding partner.

Whilst the sequences and properties of proteins discussed above relate to human proteins, the assay procedures of the invention may be practised on samples arising from other species. Especially in this context, references to proteins herein should be understood to include proteins having a degree of homology of at least 60% with the given amino acid sequences irrespective of any modifications of said amino acids. When determining homology, modified amino acids such as phosphorylated, acetylated, amidated, methylated, glycosylated or lipidated derivatives of an amino acid should thus be considered to be the same as the amino acid without any such modification. Such peptides may be derived from similar proteins from other species, e.g. other mammals such as mouse, rabbit, guinea pig, pig, or cow or may be entirely or predominantly of synthetic origin.

The degree of homology may be advantageously be at least 65%, or at least 70%. Under certain circumstances, it is advantageous that the degree of homology is even higher such as at least 80% or at least 90%. Other DNA sequences which encode substantially the same amino acid sequence as a gene encoding a marker protein, i.e. a marker gene, may be used in the practice of the present invention. These include, but are not limited to, allelic genes and homologous genes from other species.

Nucleic acid fragments comprising a nucleotide sequence which codes for a protein described above or a peptide derived from it as well as nucleic acid fragments which hybridise with these nucleic acid fragments or a part thereof under stringent hybridisation conditions, e.g. 5 mM monovalent ions (0.1× SSC), neutral pH and 65° C. are important aspects of the invention. The term “highly stringent”, when used in conjunction with hybrisidation conditions, is as defined in the art, i.e. 5-10° C. under the melting point Tₘ, cf, Sambrook et al, 1989, pages 11.45-11.49.

By the term “nucleic acid” is meant a polynucleotide of high molecular weight which can occur as either DNA or RNA and may be either single-stranded or double-stranded.

Once the amino acid sequences of the proteins of utility in the present invention are known, it is possible to synthesise DNA or RNA probes which may be used for:

i) direct detection of DNA and RNA expressing said proteins on a fixed or frozen tissue section using, e.g. chromogenous, chemiluminescent or immunofluorescent techniques;

ii) polymerase chain reaction (PCR) or other amplification techniques; and

iii) locating the part or all of the gene, isogene, pseudogene or other related genes either in cDNA libraries, genomic libraries or other collections of genetic material from either the host or other animals, including man.

In another aspect, the invention relates to a binding means which specifically binds to a relevant protein or peptide or nucleic acid fragment as described above. In particular, the invention relates to an antibody which specifically binds to a relevant protein or peptide or an antigen-binding fragment thereof, i.e. a polyclonal antibody, a monoclonal antibody, chimeric antibody, single chain antibody fragment, Fab and Fab′ fragments, and an Fab expression library.

It is contemplated that both monoclonal and polyclonal antibodies will be useful in providing the basis for one or more assays to detect relevant peptides and proteins. Antibodies which are directed against epitopes that are specific for the proteins will be most useful as cross reaction will be minimised therewith.

Based upon the identification of relevant proteins described above, assay methods and kits may be produced according to standard methodology. Thus, the proteins may be obtained in purified form, either by extraction from tissues or by synthesis, and antibodies may be raised thereto or to characterising peptide sequences thereof. Standard assay formats employing such antibodies may be utilised according to the invention.

Preferred immunoassays are contemplated as including various types of enzyme linked immunoassays (ELISA), immunoblot techniques, and the like, known in the art. However, it is readily appreciated that utility is not limited to such assays, and useful embodiments including RIAs and other non-enzyme linked antibody binding assays or procedures. The proteins themselves or peptides derived from the protein sequences may be used in detecting auto-antibodies to such proteins.

Samples of the proteins described above have been subjected to trypsin digestion and the molecular weight of the resulting fragments has been determined by mass spectrometry. This provides a “fingerprint” of the protein which can be matched to date in established data bases available to those working in this field. This procedure has enabled us to identify certain of the proteins as being previously known in other contexts. No matches have been found for certain others, indicating that they have not previously been known.

The invention will be illustrated and explained further by the following description in which the Figures as follows:

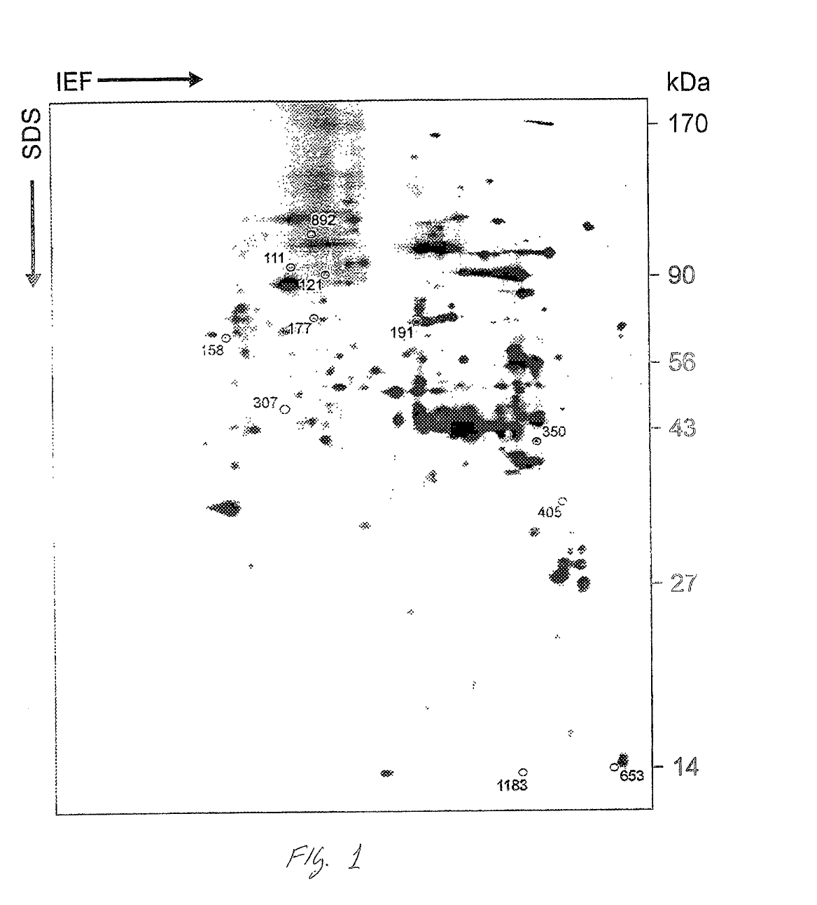

FIG. 1: Fluorograph of a two-dimensional gel electrophoresis of [³⁵S]methionine labelled endometrial proteins separated in the first dimension by isoelectric focusing (IEF; pI 3.5-7) and in the second dimension by sodium dodecyl sulphate polyacrylamide gel electrophoresis. The locations of the spots with increased synthesis in hyperplasia are indicated.

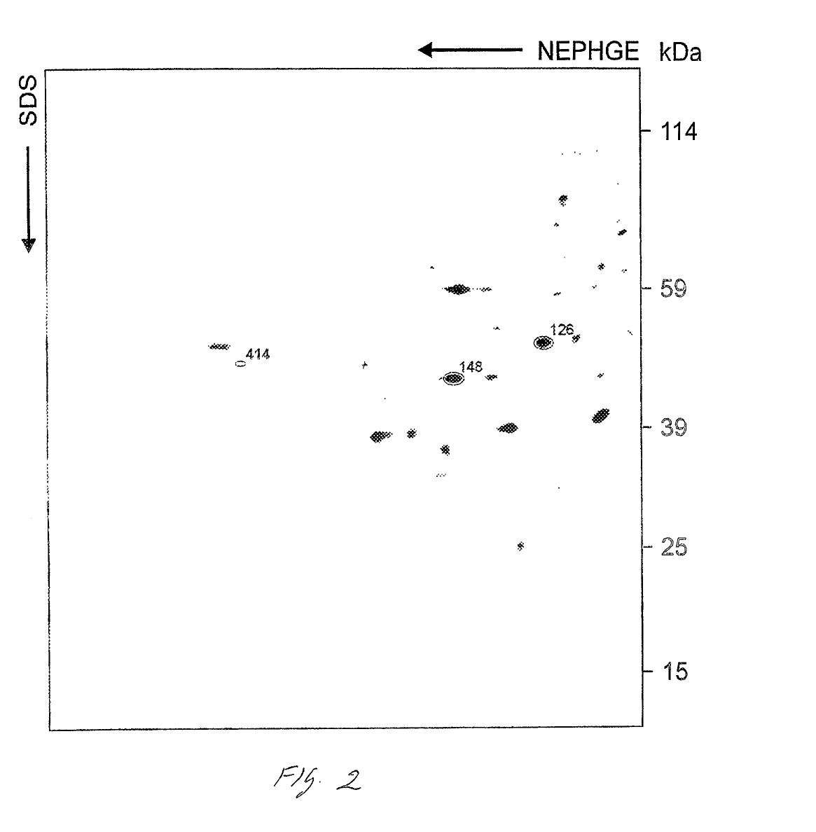

FIG. 2: Fluorograph of a two-dimensional gel electrophoresis of [³⁵S]methionine labelled endometrial proteins separated in the first dimension by non-equilibrium pH gradient gel electrophoresis (NEPHGE; pI 6.5-11) and-in the second dimension by sodium dodecyl sulphate polyacrylamide gel electrophoresis. The locations of the spots with increased synthesis in hyperplasia are indicated.

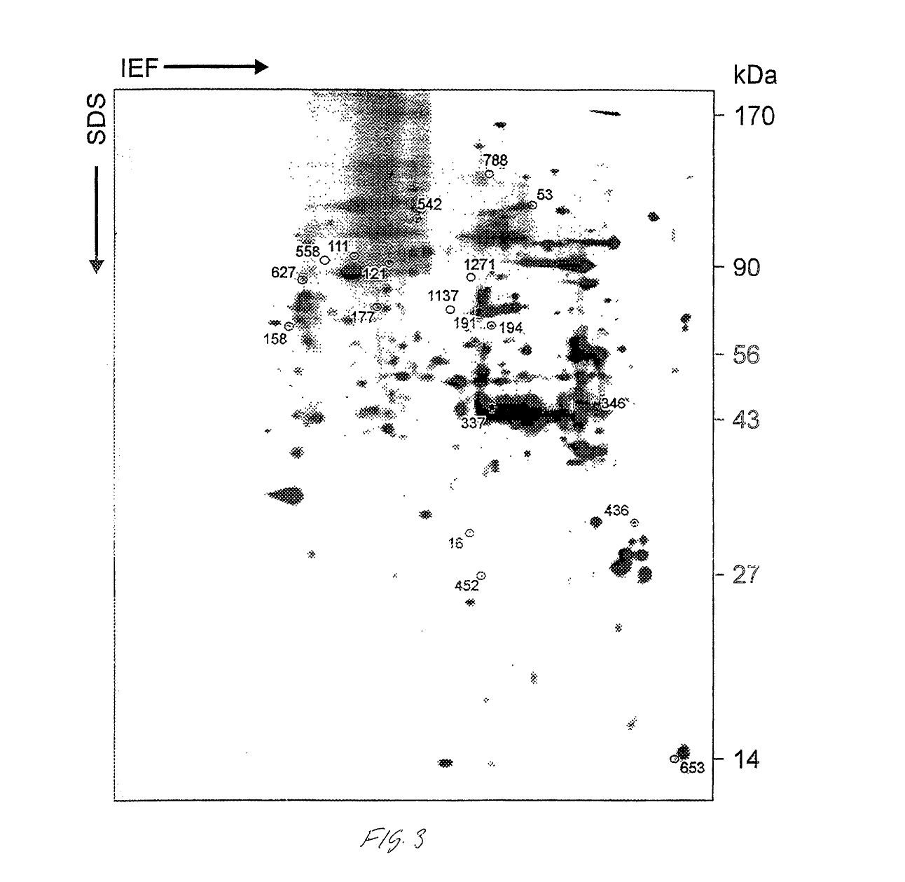

FIG. 3: Fluorograph of a two-dimensional gel electrophoresis of [³⁵S]methionine labelled endometrial proteins separated in the first dimension by isoelectric focusing (IEF; pI 3.5-7) and in the second dimension by sodium dodecyl sulphate polyacrylamide gel elctrophoresis. The locations of the spots with increased synthesis in adenocarcinoma are indicated.

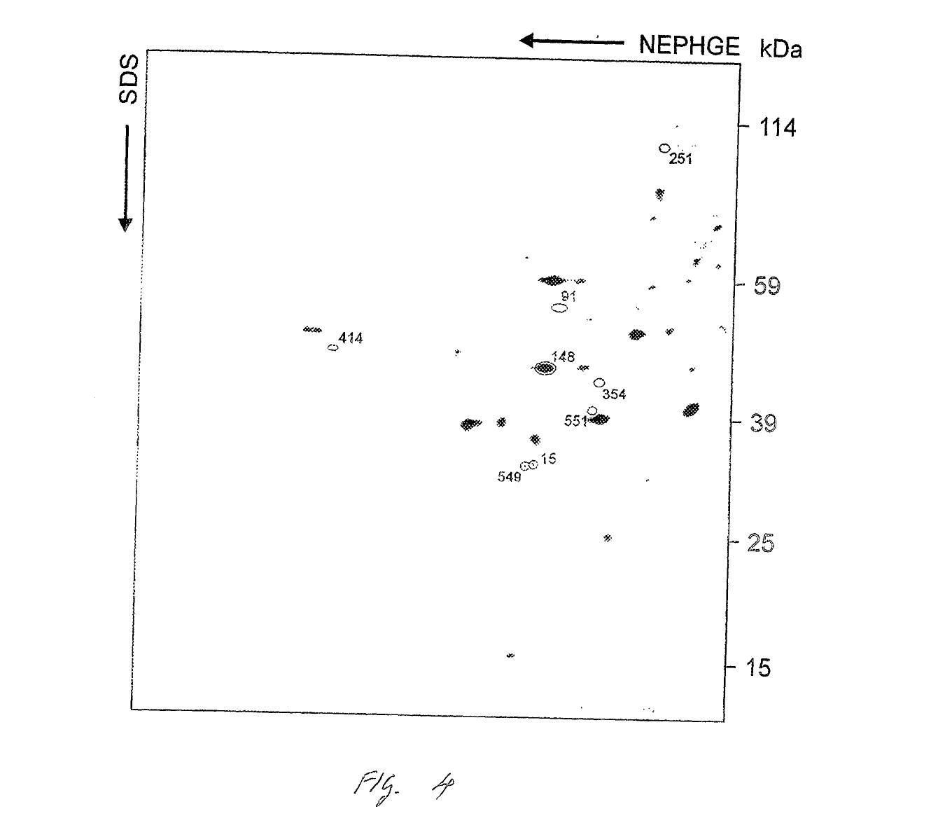

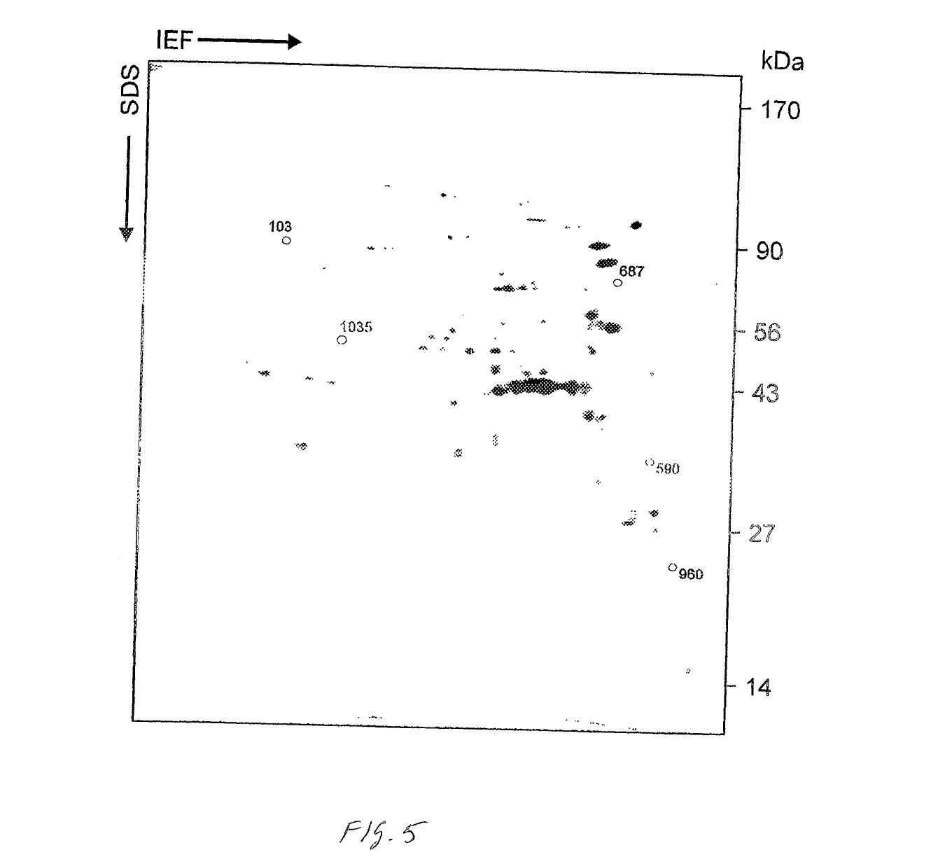

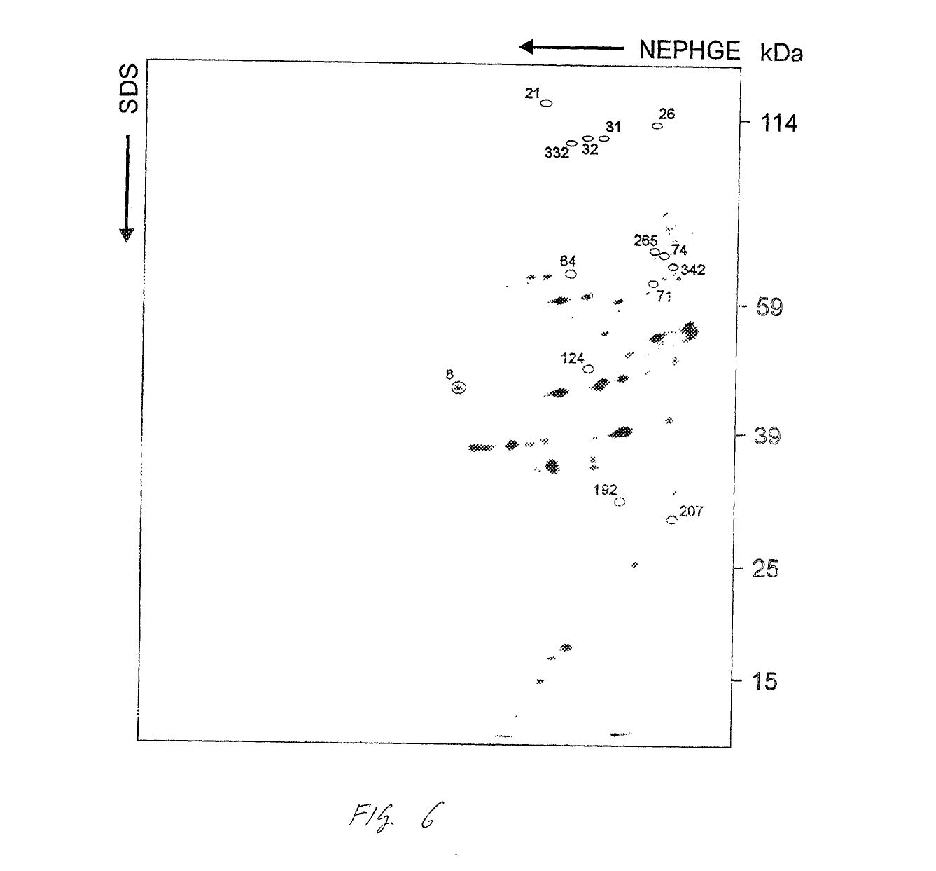

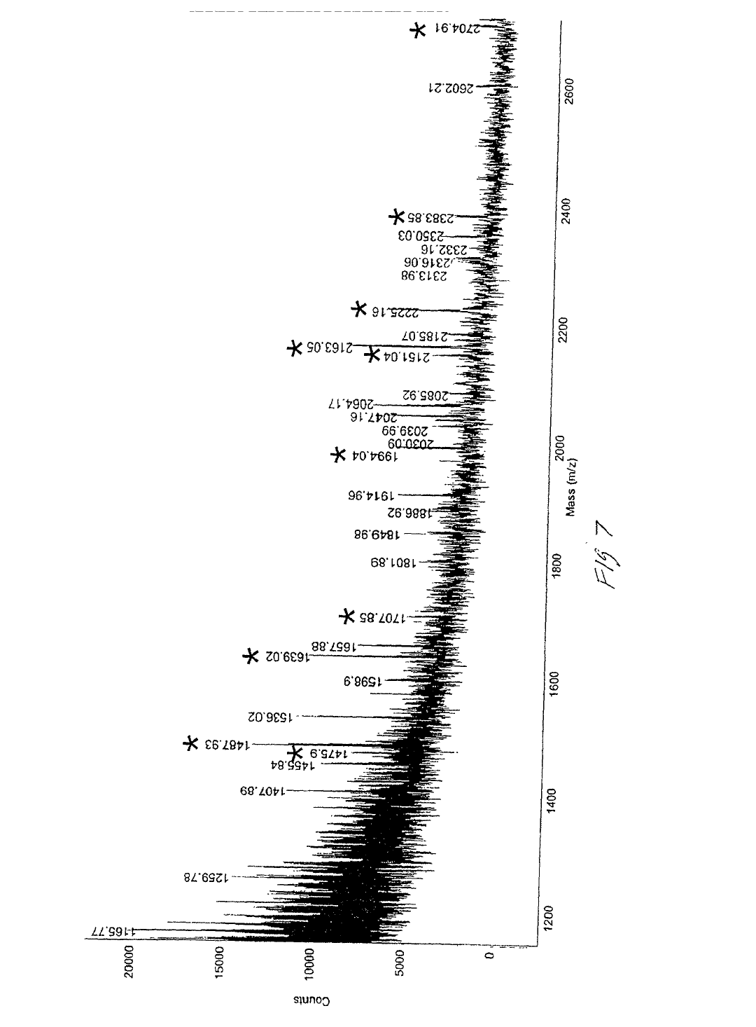

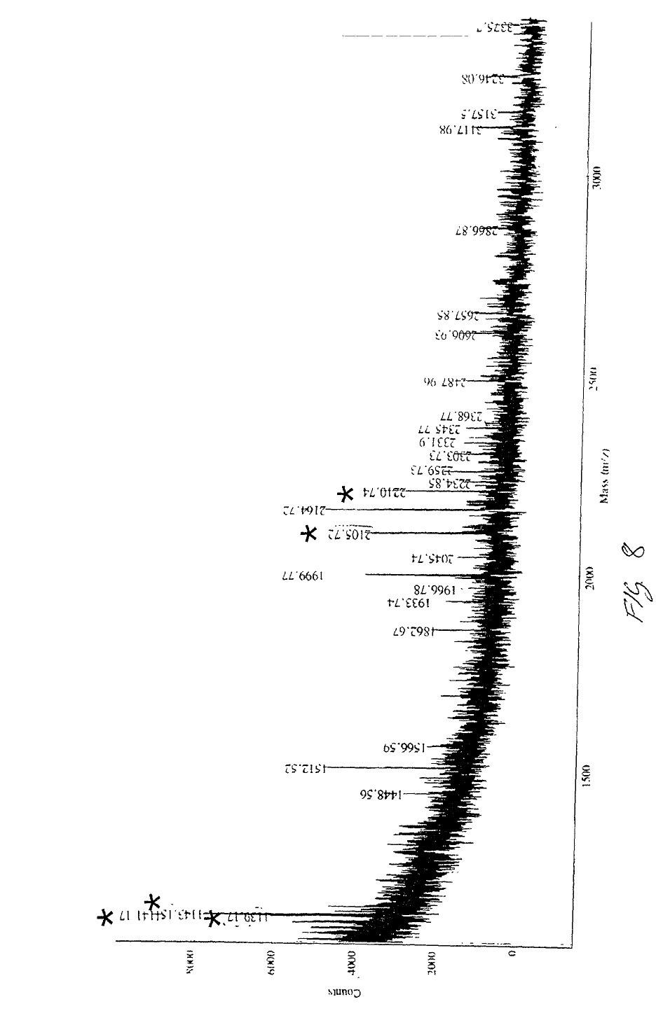

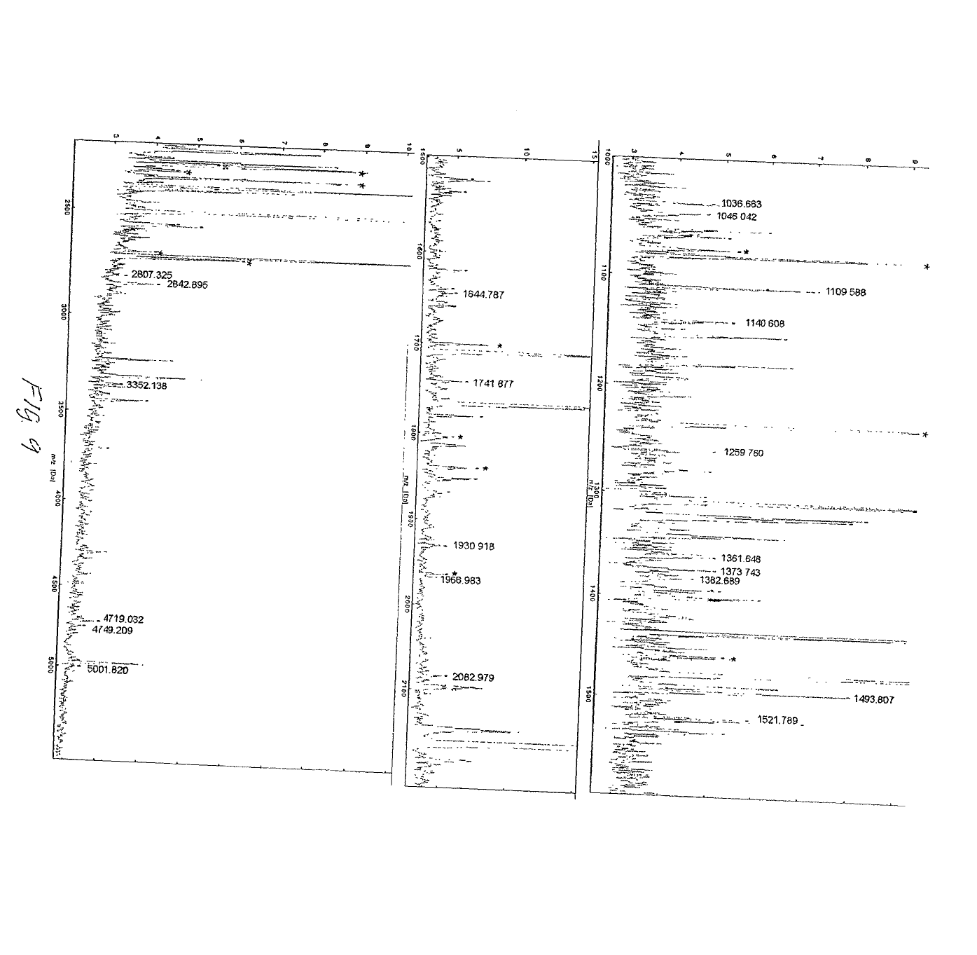

FIG. 4: Fluorograph of a two-dimensional gel electrophoresis of [³⁵S]methionine labelled endometrial proteins separated in the first dimension by non-equilibrium pH gradient gel electrophoresis (NEPHGE; pI 6.5-11) and in the second dimension by sodium dodecyl sulphate polyacrylamide gel electrophoresis. The locations of the spots with increased synthesis in adenocarcinoma are indicated.

FIG. 5: Fluorograph of a two-dimensional gel electrophoresis of [³⁵S]methionine labelled endometrial proteins separated in the first dimension by isoelectric focusing (IEF; pI 3.5-7) and in the second dimension by sodium dodecyl sulphate polyacrylamide gel elctrophoresis. The locations of the spots with increased synthesis in proliferative phase endometrium are indicated.

FIG. 6: Fluorograph of a two-dimensional gel electrophoresis of [³⁵S]methionine labelled endometrial proteins separated in the first dimension by non-equilibrium pH gradient gel electrophoresis (NEPHGE; pI 6.5-11) and in the second dimension by sodium dodecyl sulphate polyacrylamide gel electrophoresis. The locations of the spots with increased synthesis in proliferative phase endometrium are indicated.

FIG. 7: Tryptic digestion mass spectroscopic characteristics of I#350. The peaks marked with a star are not protein identification specific but represents methodologically non-specific peaks.

FIG. 8: Tryptic digestion mass spectroscopic characteristics of I#687. The peaks marked with a star are not protein identification specific but represents methodologically non-specific peaks.

FIG. 9: Tryptic digestion mass spectroscopic characteristics of N#414. The peaks marked with a star are not protein identification specific but represents methodologically non-specific peaks.

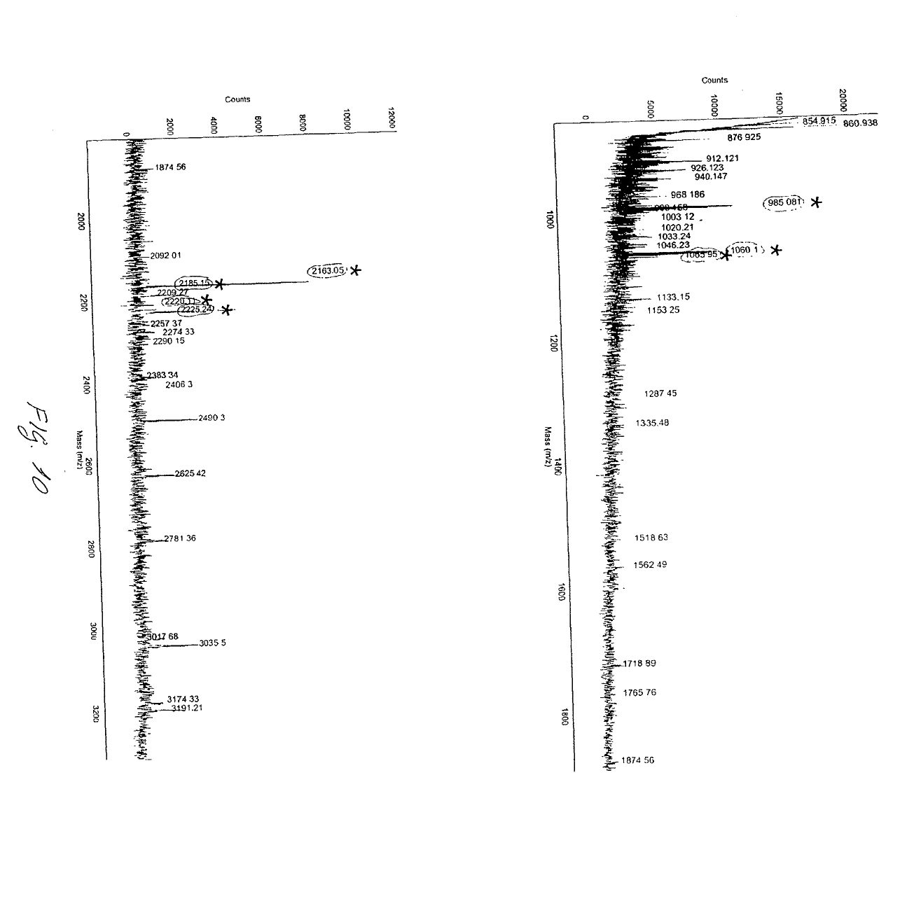

FIG. 10: Tryptic digestion mass spectroscopic characteristics of I#1035. The peaks marked with a star are not protein identification specific but represents methodologically non-specific peaks.

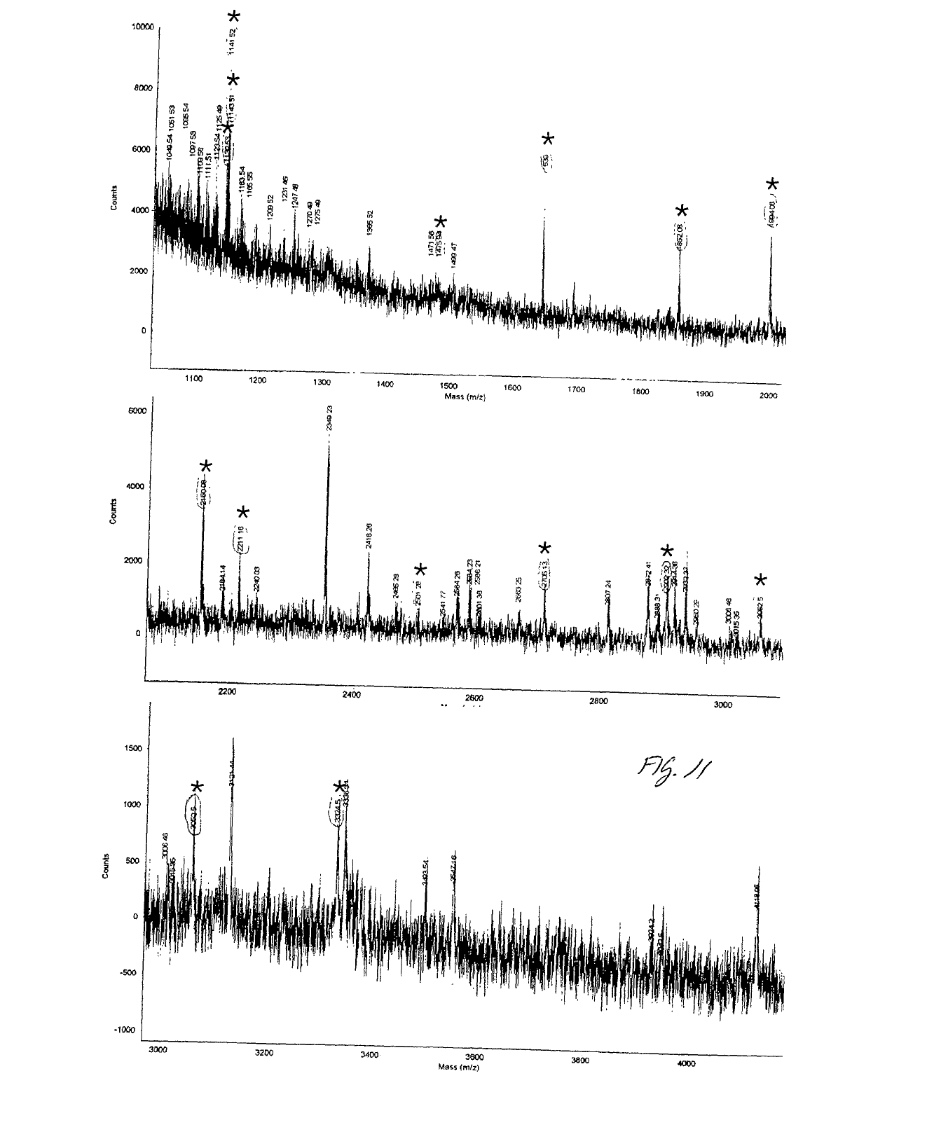

FIG. 11: Tryptic digestion mass spectroscopic characteristics of N#26. The peaks marked with a star are not protein identification specific but represents methodologically non-specific peaks.

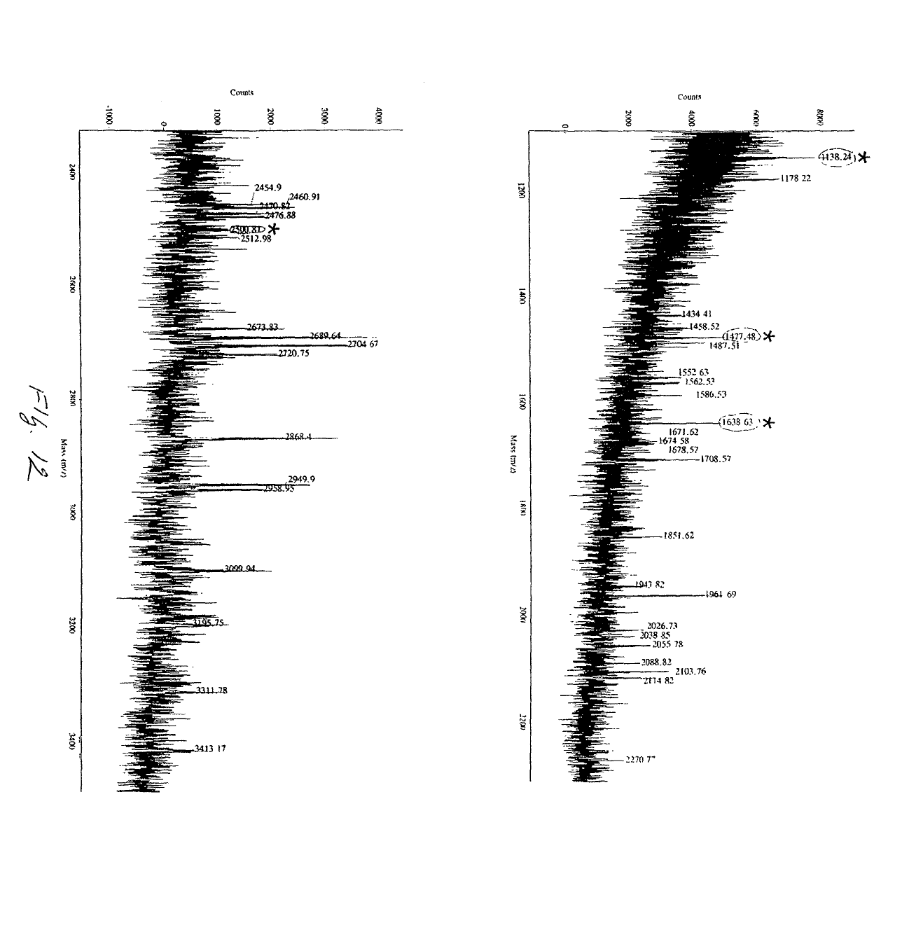

FIG. 12: Tryptic digestion mass spectroscopic characteristics of N#31+N#32. The peaks marked with a star are not protein identification specific but represents methodologically non-specific peaks.

To identify proteins expressed at an increased level in differing endometrial conditions, endometrial samples were obtained as follows.

Normal menstrual cycle samples were obtained as described in Ref. 1. Endometrial biopsies were collected from 13 pre-menopausal, regular cycling women (35-50 years old) undergoing endometrial curettage (n=1) or hysterectomy (removal of the uterus) (n=12) for a variety of medical reasons not related to abnormality or malignancy of the endometrium. None used hormone contraception. For pathological condition samples, endometrial biopsies were collected from 16 patients (41 to 79 years old) undergoing endometrial curettage (n=9) or hysterectomy (n=7) for medical reasons related to abnormality or malignancy of the endometrium.

The samples were treated as described in Ref. 1. The proteins of the endometrial biopsies were metabolically labelled with ³⁵S-methionine for 20 hours, and total cell lysates were processed for 2D gel electrophoresis, a technique in which proteins are separated in the first dimension according to the isoelectric point and in the second dimension according to the molecular weight. It was possible to study proteins with iso-electric points ranging from 3.5 to 11 and relative molecular weights ranging from 10 to 300 kDa. After electrophoresis the gels were fixed and treated for fluorography. The fluorograms of the 2D gel electrophoresis were subjected to quantitative analysis by computer-aided analysis, by which the density of each spot was quantified, the fluorogram patterns were matched i.e. numbers were assigned to each spot and the same spot was given the same number on all the fluorograms. The density (quantity synthesis) of each spot was assessed to find proteins with increased synthesis in endometrial adenocarcinoma or hyperplasia and assessed for periodic characteristics during the normal menstrual cycle to find proteins with the menstrual cycle-related synthesis.

Some of the menstrual cycle-related proteins so identified have been identified by amino acid sequence analysis (Ref.2). Selected menstrual cycle-related proteins were excised from several 2D gels, concentrated by 1D sodium dodecylsulphate polyacrylamide gel electrohoresis, and cleaved in situ by trypsin. The tryptic fragments were extracted and separated by reverse phase high pressure liquid chromatography. Finally, the partial amino-terminal amino acid sequence of selected tryptic fragments were determined for each protein. For identification the amino acid sequences of the tryptic fragments were compared to previously reported sequences by searching in databases.

The hyperplasia and adenocarcinoma associated proteins of the present invention may be sequenced and further characterised by similar methods.

Out of a total number of approximately 1,700 spots, 14 spots were found to have increased synthesis in hyperplasia. The locations of these are shown in FIGS. 1 and 2. Some 27 spots had increased synthesis in adenocarcinoma. The locations of these are shown in FIGS. 3 and 4. The information obtained from the 2D-gel electrophoresis with respect to the isoelectric point (pI) and the molecular weight (MW) of the spots with increased synthesis in hyperplasia is given in Table 1, and the spots with increased synthesis in adenocarcinoma are listed in Table 2. Eight spots had increased expression in both hyperplasia and adenocarcinoma. Based on subjective evaluation, preferred subgroups of spots were selected with increased synthesis in hyperplasia and in adenocarcinoma, respectively. The preferred subgroup of spots with increased synthesis in hyperplasia were selected as being the spots showing the highest relative increase in expression in hyperplasia as compared to the samples obtained from women during the normal mentrual cycle and women with irregular proliferative phase endometrium. Similarly, the preferred subgroup of spots with increased synthesis in adenocarcinoma were selected as the spots showing the highest relative increase in expression in adenocarcinoma as compared to the samples obtained from women during the normal menstrual cycle and women with irregular proliferative phase endometrium. The preferred subgroup of 7 spots with increased synthesis in hyperplasia is given in Table 3, and the preferred subgroup of 12 spots with increased synthesis in adenocarcinoma is given in Table 4.

TABLE 1
Endometrial proteins with increased synthesis in hyperplasia

| Match # | pI | MW (kDa) |
|---|---|---|
| I#111 | 6.7 | 91 |
| I#121 | 6.6 | 90 |
| I#158 | 6.9 | 64 |
| I#177 | 6.6 | 67 |
| I#191 | 6.3 | 66 |
| I#307 | 6.8 | 46 |
| I#350 | 5.7 | 41 |
| I#405 | 5.5 | 35 |
| I#653 | 5.3 | 13 |
| I#892 | 6.6 | 101 |
| I#1183 | 5.8 | 14 |
| N#126 | 7.4 | 51 |
| N#148 | 8.2 | 44 |
| N#414 | 9.5 | 48 |

TABLE 2
Endometrial proteins with increased synthesis in adenocarcinoma

| Match # | pI | MW (kDa) |
|---|---|---|
| I#16 | 6.3 | 32 |
| I#53 | 6.0 | 109 |
| I#111 | 6.7 | 91 |
| I#121 | 6.6 | 90 |
| I#158 | 6.9 | 64 |
| I#177 | 6.6 | 67 |
| I#191 | 6.3 | 66 |
| I#194 | 6.2 | 62 |
| I#337 | 6.2 | 45 |
| I#346 | 5.7 | 45 |
| I#436 | 5.4 | 33 |
| I#452 | 6.3 | 27 |
| I#542 | 6.5 | 103 |
| I#558 | 6.8 | 90 |
| I#627 | 6.9 | 78 |
| I#653 | 5.3 | 13 |
| I#788 | 6.2 | 130 |
| I#1137 | 6.3 | 66 |
| I#1271 | 6.3 | 73 |
| N#15 | 8.3 | 32 |
| N#91 | 8.1 | 55 |
| N#148 | 8.2 | 44 |
| N#251 | 6.6 | 111 |
| N#354 | 7.7 | 43 |
| N#414 | 9.5 | 48 |
| N#549 | 8.3 | 32 |
| N#551 | 7.7 | 39 |

TABLE 3
Preferred endometrial proteins with increased synthesis in hyperplasia

| Match # | pI | MW (kDa) |
|---|---|---|
| I#111 | 6.7 | 91 |
| I#158 | 6.9 | 64 |
| I#191 | 6.3 | 66 |
| I#350 | 5.7 | 41 |
| I#405 | 5.5 | 35 |
| I#653 | 5.3 | 13 |
| I#892 | 6.6 | 101 |

TABLE 4
Preferred endometrial proteins with increased synthesis in
adenocarcinoma

| Match # | pT | MW (kDa) |
|---|---|---|
| I#111 | 6.7 | 91 |
| I#158 | 6.9 | 64 |
| I#191 | 6.3 | 66 |
| I#194 | 6.2 | 62 |
| I#337 | 6.2 | 45 |
| I#346 | 5.7 | 45 |
| I#452 | 6.3 | 27 |
| I#627 | 6.9 | 78 |
| I#653 | 5.3 | 13 |
| N#91 | 8.1 | 55 |
| N#354 | 7.7 | 43 |
| N#551 | 7.7 | 39 |

Out of the total number of approximately 1,700 spots, 135 had a menstrual cycle-related expression. These 135 spots had maximal expression as follows: 61 spots in proliferative endometrium, 29 spots in interval phase endometrium, 41 in secretory phase endometrium and 4 in late secretory/menstrual phase endometrium. The information obtained from the 2D-gel electrophoresis with respect to the isoelectric point (pI) and the molecular weight (MW) of a preferred subgroup of these spots which show increased synthesis in proliferative phase endometrium are given in Table 5 and their positions are indicated in FIGS. 5 and 6.

TABLE 5
Endometrial proteins with menstrual cycle-related expression
Maximal expression in proliferative phase endometrium

| Match # | pI | MW (kDa) |
|---|---|---|
| I#103 | 6.9 | 86 |
| I#590 | 5.4 | 34 |
| I#687 | 5.6 | 67 |
| I#960 | 5.3 | 23 |
| I#1035 | 6.8 | 52 |
| N#8 | 8.7 | 47 |
| N#21 | 8.2 | 138 |
| N#26 | 6.5 | 124 |
| N#31 | 7.7 | 119 |
| N#32 | 7.8 | 119 |
| N#64 | 8.1 | 66 |
| N#71 | 7.1 | 59 |
| N#74 | 6.8 | 66 |
| N#124 | 7.9 | 48 |
| N#192 | 7.7 | 31 |
| N#207 | 6.8 | 29 |
| N#265 | 7.2 | 70 |
| N#332 | 8.0 | 119 |
| N#342 | 6.7 | 62 |

Fluorographs of gels exemplifying those upon which the identifications given in Tables 1 to 5 above are based appear in FIGS. 1 to 6.

The proteins described above may be further characterised by partial amino acid sequence analysis as described in Ref. 2, or by the more sensitive technique of mass spectrometric peptide mapping. By way of example, we have identified the proteins for which previously given names, data-base accession numbers and amino acid sequences are given in Table 6. Mass spectroscopic characteristics of tryptic digests of further proteins are shown in FIGS. 7 to 13 which have not matches to any known protein. These proteins can be sequenced by known techniques and are included per se within the scope of the invention.

TABLE 6

| | Name | |
|---|---|---|
| Match # | ID | Amino Acid Sequence |
| I#191 | Human heat | MAKAAAIGID LGTTYSCVGV FQHGKVEIIA |
| And | shock 70 kD | NDQGNRTTPS YVAFTDTERL IGDAAKNQVA |
| I#1137 | protein 1 | LNPQNTVFDA KRLIGRKFGD PVVQSDMKHW |
| SEQ ID | P08107 | PFQVINDGDK PKVQVSYKGE TKAFYPEEIS |
| No.1 | | SMVLTKMKET AEAYLGYPVT NAVTTVPAYF |
| | | NDSQRQATKD AGVIAGLNVL RIINEPTAAA |
| | | IAYGLDRTGK GERNVLIFDL GGGTFDVSIL |
| | | TIDDGIFEVK ATAGDTHLGG EDFDNRLVNH |
| | | FVEEFKRKHK KDISQNKRAV RRLRTACERA |
| | | KRTLSSSTQA SLEIDSLFEG IDFYTSITRA |
| | | RFEELCSDLF RSTLEPVEKA LRDAKLDKAQ |
| | | IHDLVLVGGS TRIPKVQKLL QDFFNGRDLN |
| | | KSINPDEAVA YGAAVQAAIL MGDKSENVQD |
| | | LLLLDVAPLS LGLETACCVM TALIKRNSTI |
| | | PTKQTQIFTT YSDNQPGVLI QVYEGERAMT |
| | | KDNNLLGRFE LSGIPPAPRG VPQIEVTFDI |
| | | DANGTLNVTA TDKSTGKANK ITITNDKGRL |
| | | SKEEIERMVQ EAEKYKAEDE VQRERVSAKN |
| | | ALESYAFNMK SAVEDEGLKG KISEADKKKV |
| | | LDKCQEVISW LDANTLAEKD EFEHKRKELE |
| | | QVCNPIISGL YQGAGGPGPG GFGAQGPKGG |
| | | SGSGPTIEEV D |
| I#337 | CAMP- | ASPPACPSEE DESLKGCELY VQLHGQQVL |
| SEQ | dependent | KDCIVHLCIS KPERPMKFLR EHFEKLEKEE |
| ID | protein | NRQILARQKS NSQSDSHDEE VSPTPPNPVV |
| No.2 | kinase type | KARRRRGGVS AEVYTEEDAV SYVRKVIPKD |
| | I-beta | YKTMTALAKA ISKNVLFAHL DDNERSDIFD |
| | regulatory | AMFPVTHIAG ETVIQQGNEG DNFYVVDQGE |
| | chain | VDVYVNGEWV TNISEGGSFG ELALIYGTPR |
| | P31321 | AATVKAKTDL KLWGIDRDSY RRILMGSTLR |
| | | KRKMYEEFLS KVSILESLEK WERLTVADRL |
| | | EPVQFEDGEK IVVQGEPGDD FYIITEGTAS |
| | | VLQRRSPNEE YVEVGRLGPS DYFGEIALLL |
| | | NRPRAATVVA RGPLKCVKLD RPRFERVLGP |
| | | CSEILKRNIQ RYNSFISLTV |
| I#346 | Vimentin | STRSVSSSSY RRMFGGPGTA SRPSSSRSYV |
| And | | TTSTRTYSLG SALRPSTSRS LYASSPGGVY |
| I#405 | P08670 | ATRSSAVRLR SSVPGVRLLQ DSVDFSLADA |
| SEQ ID | | INTEFKNTRT NEKVELQELN DRFANYIDKV |
| No.3 | | RFLEQQNKIL LAELEQLKGQ GKSRLGDLYE |
| | | EEMRELRRQV DQLTNDKARV EVERDNLAED |
| | | IMRLREKLQE EMLQREEAEN TLQSFRQDVD |
| | | NASLARLDLE RKVESLQEEI AFLKKLEEEE |
| | | IQELQAQIQE QHVQIDVDVS KPDLTAALRD |
| | | VRQQYESVAA KNLQEAEEWY KSKFADLSEA |
| | | ANRNNDALRQ AKQESTEYRR QVQSLTCEVD |
| | | ALKGTNESLE RQMREMEENF AVEAANYQDT |
| | | IGRLQDEIQN MKEEMARHLR EYQDLLNVKM |
| | | ALDIEIATYR KLLEGEESRI SLPLPNFSSL |
| | | NLRETNLDSL PLVDTHSKRT FLIKTVETRD |
| | | GQVINETSQH HDDLE |
| I#452 | Heat Shock 27 | MTERRVPFSL LRGPSWDPFR DWYPHSRLFD |
| | KD Protein | QAFGLPRLPE EWSQWLGGSS WPGYVRPLPP |
| SEQ ID | | AAIESPAVAA PAYSRALSRQ LSSGVSEIRH |
| No.4 | P04792 | TADRWRVSLD VNHFAPDELT VKTKDGVVEI |
| | | TGKHEERQDE HGYISRCFTR KYTLPPGVDP |
| | And | TQVSSSLSPE GTLTVEAPMP KLATQSNEIT |
| | Prohibitin | IPVTFESRAQ LGGRSCKIR |
| | P35232 | MAAKVFESIG KFGLALAVAG GVVNSALYNV |
| | in | DAGHRAVIFD RFRGVQDIVV GEGTHFLIPW |
| | admixture) | VQKPIIFDCR SRPRNVPVIT GSKDLQNVNI |
| | | TLRILFRPVA SQLPRIFTSI GEDYDERVLP |
| | | SITTEILKSV VARFDAGELI TQRELVSRQV |
| | | SDDLTERAAT FGLILDDVSL THLTFGKEFT |
| | | EAVEAKQVAQ QEAERARFVV EKAEQQKKAA |
| | | IISAEGDSKA AELIANSLAT AGDGLIELRK |
| | | LEAAEDIAYQ LSRSRNITYL PAGQSVLLQL PQ |
| I#436 | Tropomyosin | MDAIKKKMQM LKLDKENALD RAEQAEADKK |
| And | fibroblast | AAEDRSKQLE DELVSLQKKL KGTEDELDKY |
| I#590 | isoform TM3 | SEALKDAQEK LELAEKKATD AEADVASLNR |
| SEQ ID | P09494 | RIQLVEEELD RAQERLATAL QKLEEAEKAA |
| No.5 | | DESERGMKVI ESRAQKDEEK MEIQEIQLKE |
| | | AKHIAEDADR KYEEVARKLV IIESDLERAE |
| | | ERAELSEGQV RQLEEQLRIM DQTLKALMAA |
| | | EDKYSQKEDR YEEEIKVLSD KLKEAETRAE |
| | | FAERSVTKLE KSIDDLEEKV AHAKEENLSM |
| | | HQMLDQTLLE LNNM |
| I#627 | Serotrans- | MRLAVGALLV CAVLGLCLAV PDKTVRWCAV |
| SEQ ID | ferrin | SEHEATKCQS FRDHMKSVIP SDGPSVACVK |
| No.6 | precursor | KASYLDCIRA IAANEADAVT LDAGLVYDAY |
| | P02787 | LAPNNLKPVV AEFYGSKEDP QTFYYAVAVV |
| | | KKDSGFQMNQ LRGKKSCHTG LGRSAGWNIP |
| | | IGLLYCDLPE PRKPLEKAVA NFFSGSCAPC |
| | | ADGTDFPQLC QLCPGCGCST LNQYFGYSGA |
| | | FKCLKDGAGD VAFVKHSTIF ENLANKADRD |
| | | QYELLCLDNT RKPVDEYKDC HLAQVPSHTV |
| | | VARSMGGKED LIWELLNQAQ EHFGKDKSKE |
| | | FQLFSSPHGK DLLFKDSAHG FLKVPPRMDA |
| | | KMYLGYEYVT AIRNLREGTC PEAPTDECKP |
| | | VKWCALSHHE RLKCDEWSVN SVGKIECVSA |
| | | ETTEDCIAKI MNGEADAMSL DGGFVYIAGK |
| | | CGLVPVLAEN YNKSDNCEDT PEAGYFAVAV |
| | | VKKSASDLTW DNLKGKKSCH TAVGRTAGWN |
| | | IPMGLLYNKI NHCRFDEFFS EGCAPGSKKD |
| | | SSLCKLCMGS GLNLCEPNNK EGYYGYTGAF |
| | | RCLVEKGDVA FVKHQTVPQN TGGKNPDPWA |
| | | KNLNEKDYEL LCLDGTRKPV EEYANCHLAR |
| | | APNHAVVTRK DKEACVHKIL RQQQHLFGSN |
| | | VTDCSGNFCL FRSETKDLLF RDDTVCLAKL |
| | | HDRNTYEKYL GEEYVKAVGN LRKCSTSSLL EACTFRRP |
| N#8 | 47 KD Heat | MRSLLLGTLC LLAVALAAEV KKPVEAAAPG |
| SEQ ID | Shock Protein | TAEKLSSKAT TLAEPSTGLA FSLYQAMAKD |
| No.7 | Precursor | QAVENILVSP VVVASSLGLV SLGGKATTAS |
| | P29043 | QAKAVLSAEQ LRDEEVHAGL GELLRSLSNS |
| | | TARNVTWKLG SRLYGPSSVS FADDFVRSSK |
| | | QHYNCEHSKI NFPDKRSALQ SINEWAAQTT |
| | | DGKLPEVTKD VERTDGALLV NANFFKPHWD |
| | | EKFHHKMVDN RGFMVTRSYT VGVTMMHRTG |
| | | LYNYYDDEKE KLQLVEMPLA HKLSSLIILM |
| | | PHHVEPLERL EKLLTKEQHK IWMGKMQKKA |
| | | VAISLPKGVV EVTHDLQKHL AGLGLTEAID |
| | | KNKADLSRMS GKKDLYLASV FHATAFELDT |
| | | DGNPFDQDIY GREELRSPKL FYADHPFIFL |
| | | VRDTQSGSLL FIGRLVRLKG DKMRDEL |
| N#124 | Ubiquinol- | MKLLTRAGSF SRFYSLKVAP KVKATAAPAG |
| SEQ ID | cytochrom C | APPQPQDLEF TKLPNGLVIA SLENYSPVSR |
| No.8 | reductase | IGLFIKAGSR YEDFSNLGTT HLLRLTSSLT |
| | complex core | TKGASSFKIT RGIEAVGGKL SVTATRENMA |
| | protein 2 | YTVECLRGDV DILMEFLLNV TTAPEFRRWE |
| | precursor | VADLQPQLKI DKAVAFQNPQ THVIENLHAA |
| | P22695 | AYQNALANPL YCPDYRIGKV TSEELHYFVQ |
| | | NHFTSARMAL IGLGVSHPVL KQVAEQFLNM |
| | | RGGLGLSGAK ANYRGGEIRE QNGDSLVHAA |
| | | FVAESAVAGS AEANAFSVLQ HVLGAGPHVK |
| | | RGSNTTSHLH QAVAKATQQP FDVSAFNASY |
| | | SDSGLFGIYT ISQATAAGDV IKAAYNQVKR |
| | | IAQGNLSNTD VQAAKNKLKA GYLMSVESSE |
| | | CFLEEVGSQA LVACSYMPPS TVLQQIDSVA |
| | | NADIINAAKK FVSGQKSMAA SGNLGHTPFV DEL |
| N#126 | Alpha Enolase | SILKIHAREI FDSRGNPTVE VDLFTSKGLF |
| | | RAAVPSGAST GIYEALELRD NDKTRYMGKG |
| SEQ ID | P06733 | VSKAVEHINK TIAPALVSKK LNVTEQEKID |
| No.9 | | KLMIEMDGTE NKSKFGANAI LGVSLAVCKA |
| | | GAVEKGVPLY RHIADLAGNS EVILPVPAFN |
| | | VINGGSHAGN KLAMQEFMIL PVGAANFREA |
| | | MRIGAEVYHN LKNVIKEKYG KDATNVGDEG |
| | | GFAPNILENK EGLELLKTAI GKAGYTDKVV |
| | | IGMDVAASEF FRSGKYDLDF KSPDDPSRYI |
| | | SPDQLADLYK SFIKDYPVVS IEDPFDQDDW |
| | | GAWQKFTASA GIQVVGDDLT VTNPKRIAKA |
| | | VNEKSCNCLL LKVNQIGSVT ESLQACKLAQ |
| | | ANGWGVMVSH RSGETEDTFI ADLVVGLCTG |
| | | QIKTGAPCRS ERLAKYNQLL RIEEELGSKA |
| | | KPAGRNFRNP LAK |
| N#148 | Phospho- | SLSNKLTLDK LDVKGKRVVM |
| | glycerate | RVDFNVPMKNNQITNNQRIK AAVPSIKFCL |
| | kinase 1 | DNGAKSVVLM |
| SEQ ID | P00558 | SHLGRPDGVP MPDKYSLEPV AVELKSLLGK |
| No.10 | | DVLFLKDCVG PEVEKACANP AAGSVTLLEN |
| | | LRFHVEEEGK GKDASGNKVK EPAKIEAFR |
| | | ASLSKLGDVY VNDAFGTAHR AHSSMVGVNL |
| | | PQKAGGFLMK KELNYFAKAL ESPERPFLAI |
| | | LGGAKVADKI QLINNMLDKV NEMIIGGGMA |
| | | FTFLKVLNNM EIGTSLFDEE GAKIVKDLMS |
| | | KAEKNGVKIT LPVDFVTADK FDENAKTGQA |
| | | TVASGIPAGW MGLDCGPESS KKYAEAVTRA |
| | | KQIVWNGPVG VFEWEAFARG TKALMDEVVK |
| | | ATSRGCITII GGGDTATCCA KWNTEDKVSH |
| | | VSTGGGASLE LLEGKVLPGV DALSNIL |
| N#207 | Triose- | MAPSRKFFVG GNWKMNGRKQ SLGELIGTLN |
| SEQ ID | phosphat | AAKVPADTEV VCAPPTAYID FARQKLDPKI |
| No.11 | isomerase | AVAAQNCYKV TNGAFTGEIS PGMIKDCGAT |
| | ISHUT | WVVLGHSERR HVFGESDELI GQKVAHALAE |
| | S29743 | GLGVIACIGE KLDEREAGIT EKVVFEQTKV |
| | | IADNVKDWSK VVLAYEPVWA IGTGKTATPQ |
| | | QAQEVHEKLR GWLKSNVSDA VAQSTRIIYG |
| | | GSVTGATCKE LASQPDVDGF LVGGASLKPE |
| | | FVDIINAKQ |
| N#332 | Hypo-thetical | PVPLSFLSTV CDPRVQDGAA ERTGAADGEE |
| SEQ ID | Protein | FLGGGGLPAE LFQKKVVASF PRTVLSTGMD |
| No.12 | KIAA0083 | NRYLVLAVNT VQNKEGNCEK RLVITASQSL |
| | P51530 | ENKELCILRN DWCSVPVEPG DIIHLEGDCT |
| | | SDTWIIDKDF GYLILYPDML ISGTSIASSI |
| | | RCMRRAVLSE TFRSSDPATR QMLIGTVLHE |
| | | VFQKAINNSF APEKLQELAF QTIQEIRHLK |
| | | EMYRLNLSQD EIKQEVEDYL PSFCKWAGDF |
| | | MHKNTSTDFP QMQLSLPSDN SKDNSTCNIE |
| | | VVKPMDTEES IWSPRFGLKG KIDVTVGVKI |
| | | HRGYKTKYKI MPLELKTGKE SNSIEHRSQV |
| | | VLYTLLSQER RADPEAGLLL YLKTGQMYPV |
| | | PANHLDKREL LKLRNQMAFS LFHRISKSAT |
| | | RQKTQLASLP QIIEEEKTCK YCSQIGNCAL |
| | | YSRAVEQQMD CSSVPIVMLP KIEEETQHLK |
| | | QTHLEYFSLW CLMLTLESQS KDNKKNHQNI |
| | | WLMPASEMEK SGSCIGNLIR MEHVKIVCDG |
| | | QYLHNFQCKH GAIPVTNLMA GDRVIVSGEE |
| | | RSLFALSRGY VKEINMTTVT CLLDRNLSVL |
| | | PESTLFRLDQ EEKNCDIDTP LGNLSKLMEN |
| | | TFVSKKLRDL IIDFREPQFI SYLSSVLPHD |
| | | AKDTVACILK GLNKPQRQAM KKVLLSKDYT |
| | | LIVGMPGTGK TTTICTLVRI LYACGFSVLL |
| | | TSYTHSAVDN ILLKLAKFKI GFLRLGQIQK |
| | | VHPAIQQFTE QEICRSKSIK SLALLEELYN |
| | | SQLIVATTCM GINHPIFSRK IFDFCIVDEA |
| | | SQISQPICLG PLFFSRRFVL VGDHQQLPPL |
| | | VLNREARALG MSESLFKRLE QNKSAVVQLT |
| | | VQYRMNSKIM SLSNKLTYEC KLECGSDKVA |
| | | NAVINLRHFK DVKLELEFYA DYSDNPWLMG |
| | | VFEPNNPVCF LNTDKVPAPE QVEKGGVSNV |
| | | TEAKLIVFLT SIFVKAGCSP SDIGIIAPYR |
| | | QQLKIINDLL ARSIGMVEVN TVDKYQGRDK |
| | | SIVLVSFVRS NKDGTVGELL KDWRRLNVAI |
| | | TRAKHKLILL GCVPSLNCYP PLEKLLNHLN |
| | | SEKLIIDLPS REHSSLCHIL GDFQRE |
| N#342 | Catalase | MADSRDPASD QMQHWKEQRA AQKADVLTTG |
| SEQ ID | P04040 | AGNPVGDKLN VITVGPRGPL LVQDVVFTDE |
| No.13 | | MAHFDRERIP ERVVHAKGAG AFGYFEVTHD |
| | | ITKYSKAKVF EHIGKKTPIA VRFSTVAGES |
| | | GSADTVRDPR GFAVKFYTED GNWDLVGNNT |
| | | PIFFIRDPIL FPSFIHSQKR NPQTHLKDPD |
| | | MVWDFWSLRP ESLHQVSFLF SDRGIPDGHR |
| | | HMNGYGSHTF KLVNANGEAV YCKFHYKTDQ |
| | | GIKNLSVEDA ARLSQEDPDY GIRDLFNAIA |
| | | TGKYPSWTFY IQVMTFNQAE TFPFNPFDLT |
| | | KVWPHKDYPL IPVGKLVLNR NPVNYFAEVE |
| | | QIAFDPSNMP PGIEASPDKM LQGRLFAYPD |
| | | THRHRLGPNY LHIPVNCPYR ARVANYQRDG |
| | | PMCMQDNQGG APNYYPNSFG APEQQPSALE |
| | | HSIQYSGEVR RFNTANDDNV TQVRAFYVNV |
| | | LNEEQRKRLC ENIAGHLKDA QIFTQKKAVK |
| | | NFTEVHPDYG SHIQALLDKY NAEKPKNAIH |
| | | TFVQSGSHLA AREKANL |
| N#551 | Hetero- | MEKTLETVPL ERKKREKEQF RKLFIGGLSF |
| SEQ ID | geneous | ETTEESLRNY YEQWGKLTDC VVMRDPASKR |
| No.14 | nuclear | SRGFGFVTFS SMAEVDAAMA ARPHSIDGRV |
| | ribonucleo- | VEPKRAVARE ESGKPGAHVT VKKLFVGGIK |
| | proteins | EDTEEHHLRD YFEEYGKIDT IEIITDRQSG |
| | A2/B1 | KKRCFGFVTF DDHDPVDKIV LQKYHTINGH |
| | P22626 | NAEVRKALSR QEMQEVQSSR SGRGGNFGFG |
| | | DSRGGGGNFG PGPGSNFRGG SDGYGSGRGF |
| | | GDGYNGYGGG PGGGNFGGSP GYGGGRGGYG |
| | | GGGPGYGNQG GGYGGGYDNY GGGNYGSGNY |
| | | NDFGNYNQQP SNYGPMKSGN FGGSRNMGGP |
| | | YGGGNYGPGG SGGSGGYGGR SRY |
| I#960 | Steroid | MAAEDVAATG ADPSELEGGG LLHEIFTSPL NLLLLGLCIF |
| (Prolifer | membrane | LLYKIVRGDQ PAASDSDDDE PPPLPRLKRR DFTPAELRRF |
| ative | binding | DGVQDPRILM AINGKVFDVT KGRKFYGPEG PYGVFAGRDA |
| phase | protein | SRGLATFCLD KEALKDEYDD LSDLTPAQQE TLNDWDSQFT |
| marker) | X99714 | FKYHHVGKLL KEGEEPTVYS DEEEPKDESA RKND |
| SEQ ID | | |
| No. 15 | | |
| I#177 | Heat shock | MDKGPAVGID LGTTYSCVGV FQHGKVEIIA NDQGNRTTPS |
| (Hyper- | cognate 71 KD | YVAFTDTERL IGDAAKNQVA MNPTNTVFDA KRLIGRRFDD |
| plasia & | protein | AVVQSDMKHW PFMVVNDAGR PKVQVEYKGE TKSFYPEEVS |
| Cancer | P11142 | SMVLTKMKEI AEAYLGKTVT NAVVTVPAYF NDSQRQATKD |
| Marker) | | AGTIAGLNVL RIINEPTAAA IAYGLDKKVG AERNVLIFDL |
| SEQ ID | | GGGTFDVSIL TIEDGIFEVK STAGDTHLGG EDFDNRMVNH |
| No.16 | | FIAEFKRKHK KDISENKRAV RRLRTACERA KRTLSSSTQA |
| | | SIEIDSLYEG IDFYTSITRA RFEELNADLF RGTLDPVEKA |
| | | LRDAKLDKSQ IHDIVLVGGS TRIPKIQKLL |
| | | QDFFNGKELN KSINPDEAVA YGAAVQAAIL SGDKSENVQD |
| | | LLLLDVTPLS LGIETAGGVM TVLIKRNTTI PTKQTQTFTT |
| | | YSDNQPGVLI QVYEGERAMT KDNNLLGKFE LTGIPPAPRG |
| | | VPQIEVTFDI DANGILNVSA VDKSTGKENK ITITNDKGRL |
| | | SKEDIERMVQ EAEKYKAEDE KQRDKVSSKN SLESYAFNMK |
| | | ATVEDEKLQG KINDEDKQKI LDKCNEIINW LDKNQTAEKE |
| | | EFEHQQKELE KVCNPIITKL YQSAGGMPGG MPGGFPGGGA |
| | | PPSGGASSGP TIEEVD |

ID: Accession Identification in protein or nucleotide databases (e.g. SwissProt, Protein Identification Resource (PIR) or EMBL)

The proteins of interest may be isolated from endometrial tissue or other protein sources by 2D gel electrophoresis or by using chromatographic techniques. Poly- or monoclonal antibodies towards the protein of interest can be raised, and immunoassays can be established based on such antibodies. Synthetic peptides being fragments characteristic of such proteins may be used for the same purposes. Assays may be based on more than one such protein for measurement at one time.

Ref.1 Byrjalsen et al. Hum Reprod 1995;10:13-18.

Ref.2 Byrjalsen et al., Hum Reprod 1995;10:2760-2766.

Ref.3 Julkunen et al., Endocrinology 1986;118:1782-1786.

Ref.4 : Byrjalsen et al., Obstet Gynecol 1992;79:523-528.

Ref.5 Byrjalsen et al., Hum Reprod 1992;7:1042-1047.

1. A method of characterising a biological sample comprising detecting or quantitating therein one or more proteins produced by the endometrium in increased amounts in hyperplasia or in adenocarcinoma as shown by 2D gel electrophoresis comparison of cell lysates of endometrial biopsies from normal endometrium and endometrium showing hyperplasia or adenocarcinoma, excluding variations due to the menstrual cycle, or detecting or quantitating a fragment or breakdown product thereof, or a nucleic acid coding therefor or antibodies thereto.

2. A method of characterising a biological sample comprising detecting or quantitating therein one or more proteins produced by the endometrium in increased amounts in hyperplasia or in adenocarcinoma and characterised by one of the following combinations of molecular weight and pI values:
hyperplasia

| pI | MW kDa |
|---|---|
| 6.7 | 91 |
| 6.6 | 90 |
| 6.9 | 64 |
| 6.6 | 67 |
| 6.3 | 66 |
| 6.8 | 46 |
| 5.7 | 41 |
| 5.5 | 35 |
| 5.3 | 13 |
| 6.6 | 101 |
| 5.8 | 14 |
| 7.4 | 51 |
| 8.2 | 44 |
| 9.5 | 48 |

adenocarcinoma

| pI | MW (kDa) |
|---|---|
| 6.3 | 32 |
| 6.0 | 109 |
| 6.7 | 92. |
| 6.6 | 90 |
| 6.9 | 64 |
| 6.6 | 67 |
| 6.3 | 66 |
| 6.2 | 62 |
| 6.2 | 45 |
| 5.7 | 45 |
| 5.4 | 33 |
| 6.3 | 27 |
| 6.5 | 103 |
| 6.8 | 90 |
| 6.9 | 78 |
| 5.3 | 13 |
| 6.2 | 130 |
| 6.3 | 66 |
| 6.3 | 73 |
| 8.3 | 32 |
| 8.1 | 55 |
| 8.2 | 44 |
| 6.6 | 111 |
| 7.7 | 43 |
| 9.5 | 48 |
| 8.3 | 32 |
| 7.7 | 39 |

or a fragment or breakdown product thereof, or a nucleic acid coding therefor or antibodies thereto.

3. A method as claimed in claim 1 or claim 2, wherein said protein, fragment, breakdown product, antibodies, or nucleic acid is detected in a body fluid sample.

4. An immunological binding partner specifically reactive with a protein as defined in claim 1 or claim 2 or with a fragment or breakdown product thereof or with a nucleic acid coding therefor.

5. A cell line producing a monoclonal antibody being an immunological binding partner as claimed in claim 4.

6. An assay kit for use in a method as claimed in claim 1 or claim 2, comprising an immunological binding partner as claimed in claim 4.

7. A method of characterising a biological sample comprising detecting or quantitating therein one or more proteins produced by the endometrium in increased amounts during the proliferative phase of the endometrium as shown in 2D gel electrophoresis comparison of cell lysates of endometrial biopsies from normal endometrium in its proliferative and secretory phases and characterised by one of the following combinations of molecular weight and pI values:

| pI | MW (kDa) |
|---|---|
| 6.9 | 86 |
| 5.4 | 34 |
| 5.6 | 67 |
| 5.3 | 23 |
| 6.8 | 52 |
| 8.7 | 47 |
| 8.2 | 138 |
| 6.5 | 124 |
| 7.7 | 119 |
| 7.8 | 119 |
| 8.1 | 66 |
| 7.1 | 58 |
| 6.8 | 66 |
| 7.9 | 48 |
| 7.7 | 31 |
| 6.8 | 29 |
| 7.2 | 70 |
| 8.0 | 119 |
| 6.7 | 62 |

or a fragment or breakdown product thereof, or a nucleic acid coding therefor, or an antibody thereto.

8. A method as claimed in claim 7, for detecting the phase of the endometrium.

9. A method as claimed in claim 7 or claim 8, wherein said protein, fragment, or breakdown product is detected in a body fluid sample.

10. An immunological binding partner specifically reactive with a protein as defined in claim 7 or with a fragment or breakdown product thereof or with a nucleic acid coding therefor.

11. A cell line producing a monoclonal antibody being an immunological binding partner as claimed in claim 10.

12. An assay kit for use in a method as claimed in claim 7 or claim 8, comprising an immunological binding partner as claimed in claim 10.

13. A protein produced by the endometrium in increased amounts in hyperplasia or in adenocarcinoma as shown by 2D gel electrophoresis comparison of cell lysates of endo-metrial biopsies from normal endometrium and endometrium showing hyperplasia or adenocarcinoma, excluding variations due to the menstrual cycle, and characterised by one of the following combinations of molecular weight and pI values:
hyperplasia

| pI | MW kDa |
|---|---|
| 6.7 | 91 |
| 6.6 | 90 |
| 6.9 | 64 |
| 6.8 | 46 |
| 5.7 | 41 |
| 5.3 | 13 |
| 6.6 | 101 |
| 5.8 | 14 |
| 9.5 | 48 |

adenocarcinoma

| pI | MW (kDa) |
|---|---|
| 6.3 | 32 |
| 6.0 | 109 |
| 6.7 | 91 |
| 6.6 | 90 |
| 6.9 | 64 |
| 6.2 | 62 |
| 6.5 | 103 |
| 6.8 | 90 |
| 5.3 | 13 |
| 6.2 | 130 |
| 6.3 | 66 |
| 6.3 | 73 |
| 8.3 | 32 |
| 8.1 | 55 |
| 6.6 | 111 |
| 7.7 | 43 |
| 9.5 | 48 |
| 8.3 | 32 |

14. A protein produced by the endometrium in increased amounts during the proliferative phase of the endometrium as shown in 2D gel electrophoresis comparison of cell lysates of endometrial biopsies from normal endometrium in its proliferative and secretory phases and characterised by one of the following combinations of molecular weight and pI values:

| pI | MW (kDa) |
|---|---|
| 6.9 | 86 |
| 5.6 | 67 |
| 6.8 | 52 |
| 8.2 | 138 |
| 6.5 | 124 |
| 7.7 | 119 |
| 7.8 | 119 |
| 8.1 | 66 |
| 7.1 | 58 |
| 6.8 | 66 |
| 7.7 | 31 |

15. A protein as claimed in claim 13 or claim 14, characterised by the properties:

| PI | MW (kDa) |
|---|---|
| 5.7 | 41 |
| 5.6 | 67 |
| 9.5 | 48 |
| 6.8 | 52 |
| 6.5 | 124 |
| 7.7 | 119 |
| 7.8 | 119 |

and by the respective tryptic digestion MS spectra shown in FIGS. 7 to 12.

16. The use of a protein as defined in any one of claims 1, 2 or 7 or a fragment thereof, for detecting autoantibodies to a said protein.


SEQUENCE LISTING (16 sequences)

SEQ ID NO: 1 - protein, 641 aa, Homo sapiens
MAKAAAIGIDLGTTYSCVGVFQHGKVEIIANDQGNRTTPSYVAFTDTERLIGDAAKNQVALNPQNTVFDAKRLIGRKFGDPVVQSDMKHWPFQVINDGDKPKVQVSYKGETKAFYPEEISSMVLTKMKEIAEAYLGYPVTNAVITVPAYFNDSQRQATKDAGVIAGLNVLRIINEPTAAAIAYGLDRTGKGERNVLIFDLGGGTFDVSILTIDDGIFEVKATAGDTHLGGEDFDNRLVNHFVEEFKRKHKKDISQNKRAVRRLRTACERAKRTLSSSTQASLEIDSLFEGIDFYTSITRARFEELCSDLFRSTLEPVEKALRDAKLDKAQIHDLVLVGGSTRIPKVQKLLQDFFNGRDLNKSINPDEAVAYGAAVQAAILMGDKSENVQDLLLLDVAPLSLGLETAGGVMTALIKRNSTIPTKQTQIFTTYSDNQPGVLIQVYEGERAMTKDNNLLGRFELSGIPPAPRGVPQIEVTFDIDANGILNVTATDKSTGKANKITITNDKGRLSKEEIERMVQEAEKYKAEDEVQRERVSAKNALESYAFNMKSAVEDEGLKGKISEADKKKVLDKCQEVISWLDANTLAEKDEFEHKRKELEQVCNPIISGLYQGAGGPGPGGFGAQGPKGGSGSGPTIEEVD

SEQ ID NO: 2 - protein, 380 aa, Homo sapiens
ASPPACPSEEDESLKGCELYVQLHGIQQVLKDCIVHLCISKPERPMKFLREHFEKLEKEENRQILARQKSNSQSDSHDEEVSPTPPNPVVKARRRRGGVSAEVYTEEDAVSYVRKVIPKDYKTMTALAKAISKNVLFAHLDDNERSDIFDAMFPVTHIAGETVIQQGNEGDNFYVVDQGEVDVYVNGEWVTNISEGGSFGELALIYGTPRAATVKAKTDLKLWGIDRDSYRRILMGSTLRKRKMYEEFLSKVSILESLEKWERLTVADRLEPVQFEDGEKIVVQGEPGDDFYIITEGTASVLQRRSPNEEYVEVGRLGPSDYFGEIALLLNRPRAATVVARGPLKCVKLDRPRFERVLGPCSEILKRNIQRYNSFISLTV

SEQ ID NO: 3 - protein, 465 aa, Homo sapiens
STRSVSSSSYRRMFGGPGTASRPSSSRSYVTTSTRTYSLGSALRPSTSRSLYASSPGGVYATRSSAVRLRSSVPGVRLLQDSVDFSLADAINTEFKNTRTNEKVELQELNDRFANYIDKVRFLEQQNKILLAELEQLKGQGKSRLGDLYEEEMRELRRQVDQLTNDKARVEVERDNLAEDIMRLREKLQEEMLQREEAENTLQSFRQDVDNASLARLDLERKVESLQEEIAFLKKLHEEEIQELQAQIQEQHVQIDVDVSKPDLTAALRDVRQQYESVAAKNLQEAEEWYKSKFADLSEAANRNNDALRQAKQESTEYRRQVQSLTCEVDALKGTNESLERQMREMEENFAVEAANYQDTIGRLQDEIQNMKEEMARHLREYQDLLNVKMALDIEIATYRKLLEGEESRISLPLPNFSSLNLRETNLDSLPLVDTHSKRTFLIKTVETRDGQVINETSQHHDDLE

SEQ ID NO: 4 - protein, 471 aa, Homo sapiens [sequence omitted]

SEQ ID NO: 5 - protein, 284 aa, Homo sapiens
MDAIKKKMQMLKLDKENALDRAEQAEADKKAAEDRSKQLEDELVSLQKKLKGTEDELDKYSEALKDAQEKLELAEKKATDAEADVASLNRRIQLVEEELDRAQERLATALQKLEEAEKAADESERGMKVIESRAQKDEEKMEIQEIQLKEAKHIAEDADRKYEEVARKLVIIESDLERAEERAELSEGQVRQLEEQLRIMDQTLKALMAAEDKYSQKEDRYEEEIKVLSDKLKEAETRAEFAERSVTKLEKSIDDLEEKVAHAKEENLSMHQMLDQTLLELNNM

SEQ ID NO: 6 - protein, 698 aa, Homo sapiens
MRLAVGALLVCAVLGLCLAVPDKTVRWCAVSEHEATKCQSFRDHMKSVIPSDGPSVACVKKASYLDCIRAIAANEADAVTLDAGLVYDAYLAPNNLKPVVAEFYGSKEDPQTFYYAVAVVKKDSGFQMNQLRGKKSCHTGLGRSAGWNIPIGLLYCDLPEPRKPLEKAVANFFSGSCAPCADGTDFPQLCQLCPGCGCSTLNQYFGYSGAFKCLKDGAGDVAFVKHSTIFENLANKADRDQYELLCLDNTRKPVDEYKDCHLAQVPSHTVVARSMGGKEDLIWELLNQAQEHFGKDKSKEFQLFSSPHGKDLLFKDSAHGFLKVPPRMDAKMYLGYEYVTAIRNLREGTCPEAPTDECKPVKWCALSHHERLKCDEWSVNSVGKIECVSAETTEDCIAKIMNGEADAMSLDGGFVYIAGKCGLVPVLAENYNKSDNCEDTPEAGYFAVAVVKKSASDLTWDNLKGKKSCHTAVGRTAGWNIPMGLLYNKINHCRFDEFFSEGCAPGSKKDSSLCKLCMGSGLNLCEPNNKEGYYGYTGAFRCLVEKGDVAFVKHQTVPQNTGGKNPDPWAKNLNEKDYELLCLDGTRKPVEEYANCHLARAPNHAVVTRKDKEACVHKILRQQQHLFGSNVTDCSGNFCLFRSETKDLLFRDDTVCLAKLHDRNTYEKYLGEEYVKAVGNLRKCSTSSLLEACTFRRP

SEQ ID NO: 7 - protein, 417 aa, Homo sapiens
MRSLLLGTLCLLAVALAAEVKKPVEAAAPGTAEKLSSKATTLAEPSTGLAFSLYQAMAKDQAVENILVSPVVVASSLGLVSLGGKATTASQAKAVLSAEQLRDEEVHAGLGELLRSLSNSTARNVTWKLGSRLYGPSSVSFADDFVRSSKQHYNCEHSKINFPDKRSALQSINEWAAQTTDGKLPEVTKDVERTDGALLVNAMFFKPHWDEKFHHKMVDNRGFMVTRSYTVGVTMMHRTGLYNYYDDEKEKLQLVEMPLAHKLSSLIILMPHHVEPLERLEKLLTKEQLKIWMGKMQKKAVAISLPKGVVEVTHDLQKHLAGLGLTEAIDKNKADLSRMSGKKDLYLASVFHATAFELDTDGNPFDQDIYGREELRSPKLFYADHPFIFLVRDTQSGSLLFIGRLVRLKGDKMRDEL

SEQ ID NO: 8 - protein, 453 aa, Homo sapiens
MKLLTRAGSFSRFYSLKVAPKVKATAAPAGAPPQPQDLEFTKLPNGLVIASLENYSPVSRIGLFIKAGSRYEDFSNLGTTHLLRLTSSLTTKGASSFKITRGIEAVGGKLSVTATRENMAYTVECLRGDVDILMEFLLNVTTAPEFRRWEVADLQPQLKIDKAVAFQNPQTHVIENLHAAAYQNALANPLYCPDYRIGKVTSEELHYFVQNHFTSARMALIGLGVSHPVLKQVAEQFLNMRGGLGLSGAKANYRGGEIREQNGDSLVHAAFVAESAVAGSAEANAFSVLQHVLGAGPHVKRGSNTTSHLHQAVAKATQQPFDVSAFNASYSDSGLFGIYTISQATAAGDVIKAAYNQVKRIAQGNLSNTDVQAAKNKLKAGYLMSVESSECFLEEVGSQALVAGSYMPPSTVLQQIDSVANADIINAAKKFVSGQKSMAASGNLGHTPFVDEL

SEQ ID NO: 9 - protein, 433 aa, Homo sapiens
SILKIHAREIFDSRGNPTVEVDLFTSKGLFRAAVPSGASTGIYEALELRDNDKTRYMGKGVSKAVEHINKTIAPALVSKKLNVTEQEKIDKLMIEMDGTENKSKFGANAILGVSLAVCKAGAVEKGVPLYRHIADLAGNSEVILPVPAFNVINGGSHAGNKLAMQEFMILPVGAANFREAMRIGAEVYHNLKNVIKEKYGKDATNVGDEGGFAPNILENKEGLELLKTAIGKAGYTDKVVIGMDVAASEFFRSGKYDLDFKSPDDPSRYISPDQLADLYKSFIKDYPVVSIEDPFDQDDWGAWQKFTASAGIQVVGDDLTVTNPKRIAKAVNEKSCNCLLLKVNQIGSVTESLQACKLAQANGWGVMVSHRSGETEDTFIADLVVGLCTGQIKTGAPCRSERLAKYNQLLRIEEELGSKAKFAGRNFRNPLAK

SEQ ID NO: 10 - protein, 417 aa, Homo sapiens
SLSNKLTLDKLDVKGKRVVMRVDFNVPMKNNQITNNQRIKAAVPSIKFCLDNGAKSVVLMSHLGRPDGVPMPDKYSLEPVAVELKSLLGKDVLFLKDCVGPEVEKACANPAAGSVILLENLRFHVEEEGKGKDASGNKVKAEPAKIEAFRASLSKLGDVYVNDAFGTAHRAHSSMVGVNLPQKAGGFLMKKELNYFAKALESPERPFLAILGGAKVADKIQLINNMLDKVNEMIIGGGMAFTFLKVLNNMEIGTSLFDEEGAKIVKDLMSKAEKNGVKITLPVDFVTADKFDENAKTGQATVASGIPAGWMGLDCGPESSKKYAEAVTRAKQIVWNGPVGVFEWEAFARGTKALMDEVVKATSRGCITIIGGGDTATCCAKWNTEDKVSHVSTGGGASLELLEGKVLPGVDALSNIL

SEQ ID NO: 11 - protein, 249 aa, Homo sapiens
MAPSRKFFVGGNWKMNGRKQSLGELIGTLNAAKVPADTEVVCAPPTAYIDFARQKLDPKIAVAAQNCYKVTNGAFTGEISPGMIKDCGATWVVLGHSERRHVFGESDELIGQKVAHALAEGLGVIACIGEKLDEREAGITEKVVFEQTKVIADNVKDWSKVVLAYEPVWAIGTGKTATPQQAQEVHEKLRGWLKSNVSDAVAQSTRIIYGGSVTGATCKELASQPDVDGFLVGGASLKPEFVDIINAKQ

SEQ ID NO: 12 - protein, 1076 aa, Homo sapiens
PVPLSFLSTVCDPRVQDGAAERTGAADGEEFLGGGGLPAELFQKKVVASFPRTVLSTGMDNRYLVLAVNTVQNKEGNCEKRLVITASQSLENKELCILRNDWCSVPVEPGDIIHLEGDCTSDTWIIDKDFGYLILYPDMLISGTSIASSIRCMRRAVLSETFRSSDPATRQMLIGTVLHEVFQKAINNSFAPEKLQELAFQTIQEIRHLKEMYRLNLSQDEIKQEVEDYLPSFCKWAGDFMHKNTSTDFPQMQLSLPSDNSKDNSTCNIEVVKPMDIEESIWSPRFGLKGKIDVTVGVKIHRGYKTKYKIMPLELKTGKESNSIEHRSQVVLYTLLSQERRADPEAGLLLYLKTGQMYPVPANHLDKRELLKLRNQMAFSLFHRISKSATRQKTQLASLPQIIEEEKTCKYCSQIGNCALYSRAVEQQMDCSSVPIVMLPKIEEETQHLKQTHLEYFSLWCLMLTLESQSKDNKKNHQNIWLMPASEMEKSGSCIGNLIRMEHVKIVCDGQYLHNFQCKHGAIPVTNLMAGDRVIVSGEERSLFALSRGYVKEINMTTVTCLLDRNLSVLPESTLFRLDQEEKNCDIDTPLGNLSKLMENTFVSKKLRDLIIDFREPQFISYLSSVLPHDAKDTVACILKGLNKPQRQAMKKVLLSKDYTLIVGMPGTGKTTTICTLVRILYACGFSVLLTSYTHSAVDNILLKLAKFKIGFLRLGQIQKVHPAIQQFTEQEICRSKSIKSLALLEELYNSQLIVATTCMGINHPIFSRKIFDFCIVDEASQISQPICLGPLFFSRRFVLVGDHQQLPPLVLNREARALGMSESLFKRLEQNKSAVVQLTVQYRMNSKIMSLSNKLTYEGKLECGSDKVANAVINLRHFKDVKLELEFYADYSDNPWLMGVFEPNNPVCFLNTDKVPAPEQVEKGGVSNVTEAKLIVFLTSIFVKAGCSPSDIGIIAPYRQQLKIINDLLARSIGMVEVNTVDKYQGRDKSIVLVSFVRSNKDGTVGELLKDWRRLNVAITRAKHKLILLGCVPSLNCYPPLEKLLNHLNSEKLIIDLPSREHESLCHILGDFQRE

SEQ ID NO: 13 - protein, 527 aa, Homo sapiens
MADSRDPASDQMQHWKEQRAAQKADVLTTGAGNPVGDKLNVITVGPRGPLLVQDVVFTDEMAHFDRERIPERVVHAKGAGAFGYFEVTHDITKYSKAKVFEHIGKKTPIAVRFSTVAGESGSADTVRDPRGFAVKFYTEDGNWDLVGNNTPIFFIRDPILFPSFIHSQKRNPQTHLKDPDMVWDFWSLRPESLHQVSFLFSDRGIPDGHRHMNGYGSHTFKLVNANGEAVYCKFHYKTDQGIKNLSVEDAARLSQEDPDYGIRDLFNAIATGKYPSWTFYIQVMTFNQAETFPFNPFDLTKVWPHKDYPLIPVGKLVLNRNPVNYFAEVEQIAFDPSNMPPGIEASPDKMLQGRLFAYPDTHRHRLGPNYLHIPVNCPYRARVANYQRDGPMCMQDNQGGAPNYYPNSFGAPEQQPSALEHSIQYSGEVRRFNTANDDNVTQVRAFYVNVLNEEQRKRLCENIAGHLKDAQIFIQKKAVKNFTEVHPDYGSHIQALLDKYNAEKPKNAIHTFVQSGSHLAAREKANL

SEQ ID NO: 14 - protein, 353 aa, Homo sapiens
MEKTLETVPLERKKREKEQFRKLFIGGLSFETTEESLRNYYEQWGKLTDCVVMRDPASKRSRGFGFVTFSSMAEVDAAMAARPHSIDGRVVEPKRAVAREESGKPGAHVTVKKLFVGGIKEDTEEHHLRDYFEEYGKIDTIEIITDRQSGKKRGFGFVTFDDHDPVDKIVLQKYHTINGHNAEVRKALSRQEMQEVQSSRSGRGGNFGFGDSRGGGGNFGPGPGSNFRGGSDGYGSGRGFGDGYNGYGGGPGGGNFGGSPGYGGGRGGYGGGGPGYGNQGGGYGGGYDNYGGGNYGSGNYNDFGNYNQQPSNYGPMKSGNFGGSRNMGGPYGGGNYGPGGSGGSGGYGGRSRY

SEQ ID NO: 15 - protein, 194 aa, Homo sapiens
MAAEDVAATGADPSELEGGGLLHEIFTSPLNLLLLGLCIFLLYKIVRGDQPAASDSDDDEPPPLPRLKRRDFTPAELRRFDGVQDPRILMAINGKVFDVTKGRKFYGPEGPYGVFAGRDASRGLATFCLDKEALKDEYDDLSDLTPAQQETLNDWDSQFTFKYHHVGKLLKEGEEPTVYSDEEEPKDESARKND

SEQ ID NO: 16 - protein, 646 aa, Homo sapiens
MSKGPAVGIDLGTTYSCVGVFQHGKVEIIANDQGNRTTPSYVAFTDTERLIGDAAKNQVAMNPTNTVFDAKRLIGRRFDDAVVQSDMKHWPFMVVNDAGRPKVQVEYKGETKSFYPEEVSSMVLTKMKEIAEAYLGKTVTNAVVTVPAYFNDSQRQATKDAGTIAGLNVLRIINEPTAAAIAYGLDKKVGAERNVLIFDLGGGTFDVSILTIEDGIFEVKSTAGDTHLGGEDFDNRMVNHFIAEFKRKHKKDISENKRAVRRLRTACERAKRTLSSSTQASIEIDSLYEGIDFYTSITRARFEELNADLFRGTLDPVEKALRDAKLDKSQIHDIVLVGGSTRIPKIQKLLQDFFNGKELNKSINPDEAVAYGAAVQAAILSGDKSENVQDLLLLDVTPLSLGIETAGGVMTVLIKRNTTIPTKQTQTFTTYSDNQPGVLIQVYEGERAMTKDNNLLGKFELTGIPPAPRGVPQIEVTFDIDANGILNVSAVDKSTGKENKITITNDKGRLSKEDIERMVQEAEKYKAEDEKQRDKVSSKNSLESYAFNMKATVEDEKLQGKINDEDKQKILDKCNEIINWLDKNQTAEKEEFEHQQKELEKVCNPIITKLYQSAGGMPGGMPGGFPGGGAPPSGGASSGPTIEEVD